(19) United States
(12) Patent Application Publication
(10) Pub. No.: US 2004/0122439 A1
(43) Pub. Date: Jun. 24, 2004

(54) Adjustable biomechanical templating & resection instrument and associated method

(76) Inventors: Kimberly A. Dwyer, Fort Wayne, IN (US); Christine Rusbarsky, Clinton, CT (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/327,187

(22) Filed: Dec. 20, 2002

(51) Int. Cl. A61B 17/58

(52) U.S. Cl. 606/102

(57) ABSTRACT

A tool (100) for aiding in the selection an appropriately sized implant (400) for use in performing joint arthroplasty is provided. The tool (100) cooperates with an anatomical joint (101) or with an image (102) of an anatomical joint (101). The tool (100) includes a body (110) for alignment with a first portion (112) of the anatomical joint (101) and a segment (114). The segment (114) is moveable with respect to the body (110). The segment (114) is aligned with a second portion (116) of the anatomical joint (101). The body (110) and segment (114) are adapted for measuring the relative positions of the first (112) and second (116) portions of the anatomical joint (101) for use in the selection an appropriately sized implant (400).

### CROSS-REFERENCE TO RELATED APPLICATIONS

Cross reference is made to the following applications: DEP 666 entitled “ALIGNMENT DEVICE FOR MODULAR IMPLANTS AND METHOD”, DEP 725 entitled “INSTRUMENT AND ASSOCIATED METHOD OF TRIALING FOR MODULAR HIP STEMS”, and DEP 5004 entitled “MODULAR HIP STEMS AND ASSOCIATED METHOD OF TRIALING” filed concurrently herewith which are incorporated herein by reference.

### TECHNICAL FIELD OF THE INVENTION

The present invention relates generally to the field of orthopaedics, and more particularly, to an implant for use in arthroplasty.

### BACKGROUND OF THE INVENTION

Patients who suffer from the pain and immobility caused by osteoarthritis and rheumatoid arthritis have an option of joint replacement surgery. Joint replacement surgery is quite common and it enables many individuals to function properly when it would not be possible otherwise to do so. Artificial joints usually comprise of metal, ceramic and/or plastic components that are fixed to existing bone.

Such joint replacement surgery is otherwise known as total joint arthroplasty. Total joint arthroplasty is a well-know surgical procedure by which a diseased and/or damaged joint is replaced with a prosthetic joint. In a typical total joint arthroplasty, the ends or distal portions of the bones adjacent to joint are resected, or a portion of the distal part of the bone is removed and the artificial joint is secured thereto.

Many designs and methods for manufacturing implantable articles, such as bone prostheses are known to exist. Such bone prostheses include components of artificial joints, such as elbows, hips, knees, and shoulders.

Currently, a major critical concern is the instability of the joint in total hip arthroplasty. Instability is associated with dislocation. Dislocation is particularly a problem in total hip arthroplasty.

Factors related to dislocation include surgical technique, implant design, implant positioning, and patient related factors. In total hip arthroplasty, implant systems address this concern by offering a series of products with a range of lateral offsets, neck lengths, head lengths, and leg lengths. The combination of these four factors affect the laxity of the soft tissue. By optimizing the biomechanics, the surgeon can provide the patient a stable hip much more resistant to dislocation.

In order to accommodate the range of patient and anthropometrics, a wide range of hip implant geometries are currently manufactured by DePuy Orthopaedics, Inc., the assignee of the current application and by other companies. In particular, the S-ROM® total hip system offered by DePuy Orthopaedics, Inc., includes four offsets, three neck lengths, four head lengths, and one leg length adjustment. The combination of all these biomechanical options is rather complex.

Currently, the surgeon utilizes a radiograph (x-ray) to assist in selecting the prosthesis for a patient with the proper lateral offsets, neck lengths, head lengths, and leg lengths. The surgeon utilizes the radiograph by overlaying a radiograph of the patient's femur and acetabulum with an acetate overlay. Each prosthesis implant corresponds to a particular acetate overlay. The surgeon picks the acetate overlay which most closely corresponds to the patient's natural femur. In addition, the surgeon may utilize a single nonadjustable instrument (for example DePuy instrument number 53-1420) during surgery to determine the head center of the exposed bone. This single instrument or femur resection template only demonstrates one biomechanical combination and may not match what has been templated for the patient on the radiograph.

A need therefore exists for a prosthetic implant with additional functionality.

### SUMMARY OF THE INVENTION

The present invention includes an instrument and an associated method. The instrument is an adjustable device that allows the user to slide the neck section in a medial lateral direction to template lateral offset. It may also slide in a proximal distal direction to the template leg length. Optionally, the instrument may slide in a diagonal direction to template either the neck length or head length or a combination thereof. The actual lateral offset, leg length and neck length may be etched onto part of the instrument to indicate their amounts and to assist in implant selection.

The present invention provides an instrument and a method for templating multiple biomechanical combinations with one instrument or tool. This templating may be achieved through multiple sliding mechanisms that allow the instrument to expand or contract to the desired lateral offset, neck length, head length, and/or leg length. The instrument may be utilized to template intraoperatively and preoperatively through the use of radiographs (x-rays).

According to one embodiment of the present invention, a tool for aiding in the selection an appropriately sized implant for use in performing joint arthroplasty is provided. The tool cooperates with an anatomical joint or with an image of an anatomical joint. The tool includes a body for alignment with a first portion of the anatomical joint and a segment. The segment is moveable with respect to the body. The segment is aligned with a second portion of the anatomical joint. The body and segment are adapted for measuring the relative positions of the first and second portions of the anatomical joint for use in the selection an appropriately sized implant.

According to another embodiment of the present invention a tool for use in measuring at least one of certain dimensions for aiding in the selection of an appropriate one of a plurality of femoral components of a hip implant for use in performing hip arthroplasty is provided. The tool cooperates with an anatomical joint or with a radiographic image of an anatomical joint. The tool includes a body for alignment with a first portion of the anatomical joint and a segment. The segment is moveable with respect to the body. The segment is aligned with a second portion of the anatomical joint. The tool is used to measure one of the certain dimensions when the body is in alignment with the first portion and when the segment is in alignment with the second portion.

According to a further embodiment of the present invention, a method for selecting one of a plurality of prosthetic components for use in the joint arthroplasty is provided. The method includes the step of obtaining a radiographic image. The method further includes the step of providing a tool including a first portion for alignment with a first portion of the radiographic image and a second portion for alignment with a second portion of the radiographic image. The first portion of the tool is moveable with respect to the second portion of the tool and the tool is used to measure at least one dimension of the radiographic image. The method also includes the steps of aligning the first portion of the tool with the first portion of the radiographic image and aligning the second portion of the tool with the second portion of the radiographic image. The method also includes the steps of utilizing the tool to measuring at least one dimension of the radiographic image and selecting one of the plurality of prosthetic components based upon the measured dimension.

The technical advantages of the present invention include the ability of the instrument and method to demonstrate a complete range of biomechanical options contemplated in total hip arthroplasty procedures. For example, according to one aspect of the present invention, the instrument may adjustably telescope in three directions to duplicate lateral offset, leg length, and neck length. Thus, the present invention provides for ranges of biomechanical options including those of lateral offset, leg length, and neck length.

Another technical advantage of the present invention includes the ability of the present invention to allow the surgeon to visualize head center and level of resection before making any cuts to the bone. For example, according to one aspect of the present invention, the template may be positioned over the radiograph or x-ray and the template may be adjusted to a position corresponding to the natural femur and acetabulum. Thus, the present invention provides for visualization of head center and levels of resection prior to making any cuts to the bone. The visualization prior to cutting may be either with the use of the instrument in radiographs or for the instrument to be utilized visually intraoperatively after the bone has been exposed prior to its resection.

Other technical advantages of the present invention will be readily apparent to one skilled in the art from the following figures, descriptions and claims.

### BRIEF DESCRIPTION OF THE DRAWINGS

For a more complete understanding of the present invention and the advantages thereof, reference is now made to the following description taken in connection with the accompanying drawings, in which:

FIG. 1 is a plan view of an adjustable template in accordance with a first embodiment of the present invention with lateral offset and leg length adjustments;

FIG. 2 is a perspective view of the body of the template of FIG. 1;

FIG. 3 is a plan view of the body of FIG. 2;

FIG. 4 is a perspective view of the first segment of the template of FIG. 1;

FIG. 5 is perspective view of the second segment of the template of FIG. 1;

FIG. 6 is a plan view of the template of FIG. 1 shown in position against the outline of a femur;

FIG. 7 is plan view of an adjustable template in accordance with another embodiment of the present invention with lateral offset, leg length, and head center adjustments;

FIG. 8 is an exploded plan view of the adjustable template of FIG. 7;

FIG. 9 is a side view of the adjustable template of FIG. 7;

FIG. 10 is a plan view of the body of the template of FIG. 7;

FIG. 11 is a plan view of a mirror image body to that of FIG. 10;

FIG. 12 is a plan view of the first segment of the template of FIG. 7;

FIG. 13 is a side view of the second segment of the template of FIG. 7;

FIG. 14 is a plan view of the second segment of FIG. 13;

FIG. 15 is a rear view of the second segment of FIG. 13;

FIG. 16 is a plan view of head of the template of FIG. 7;

FIG. 17 is an end view of the head of FIG. 16;

FIG. 18 is an exploded partial plan of another embodiment of an adjustable template in accordance with the present invention;

FIG. 19 is a cross-sectional view along the lines 19-19 of FIG. 18 in the directions of the arrows;

FIG. 20 is a plan view of a modular hip stem for use with the template of the present invention;

FIG. 21 is a plan view of a monolithic hip stem for use with the template of the present invention;

FIG. 22 is a plan view of various sizes of the hip stem of FIG. 20;

FIG. 23 is a plan view of another configuration of a modular hip stem for use with the template of FIG. 1 or the template of FIG. 7;

FIG. 24 is an exploded plan view of the modular hip stem of FIG. 23; and

FIG. 25 is a plan view of various sizes of the proximal bodies of the hip stem of FIG. 23;

FIG. 26 is a plan view of various sizes of the distal stem of the hip stem of FIG. 23;

FIG. 27 is a plan view of various sizes of the head stem of the hip stem of FIG. 23;

FIG. 28 is a flow chart of a method of performing arthroplasty according to the present invention; and

FIG. 29 is a flow chart of another method of performing arthroplasty according to the present invention.

### DETAILED DESCRIPTION OF THE INVENTION

Embodiments of the present invention and the advantages thereof are best understood by referring to the following descriptions and drawings, wherein like numerals are used for like and corresponding parts of the drawings.

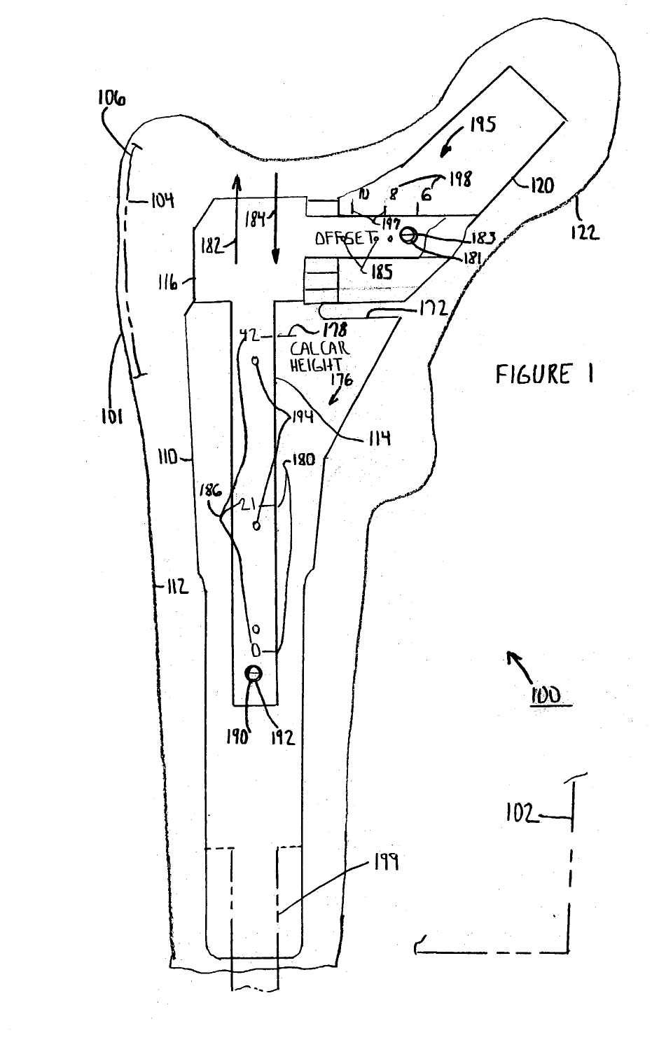

According to the present invention and referring now to FIG. 1, a tool 100 is shown. The tool 100 is utilized for aiding in the selection of an appropriately sized implant for use in performing joint arthroplasty. The tool 100 cooperates with at least one of an anatomical joint and an image thereof.

As shown in FIG. 1, the tool 100 may be used in connection with an acetate plate radiograph or an x-ray plate 102. A radiographic image 104 is formed on the acetate plate 102. The radiographic image 104 includes a 104 boundary 106 showing the outline of, for example, as shown in FIG. 1, an outline of a long bone, for example, a femur.

The tool 100 includes a body 110 for alignment with a first portion 112 of an anatomical joint 101. The tool 100 further includes a segment 114. The segment 114 is movable in respect to the body 110. The segment 114 is utilized for alignment with a second portion 116 of the anatomical joint 101. The body 110 and the segment 114 are utilized for measuring the relative position of the first portion 112 and the second portion 116 of the anatomical joint 101 for use in selection of an appropriately sized implant.

As shown in FIG. 1, the tool 100 may also include a second segment 120. The second segment 120 maybe moveable with respect to one of the body 110 and the first segment 114. The second segment 120 is utilized for alignment with a third portion 122 of the anatomical joint 101.

The tool 100, including the body 110, the first segment 114 and the second segment 120, may be made of any suitable durable material and may, for example, be made of metal and/or a plastic. Preferably, if utilized to select an implant by visual observation against an exposed femur during an operation, the tool 110 is preferably made of a sterilizable material.

The tool 100, including the body 110, the first segment 114 and the second segment 120, may have any suitable size and shape suitable for the functioning of the tool 100 to replicate and be utilized to select the appropriate prosthesis. Preferably, and as is shown in FIG. 1, when the tool 100 is utilized with a radiograph 102, the tool 100 may have the same size outline as that of the corresponding implant if the radiograph is a full scale radiograph. It should be appreciated that, if the radiograph is of reduced size or enlarged size, the tool 100 should be correspondingly reduced or enlarged to correspond to the scaling of the radiographic image. Further, when the tool 100 is utilized intraoperatively with a visual observation against the actual femur and implant, the tool 100 should have the same size and shape as that of the corresponding implant.

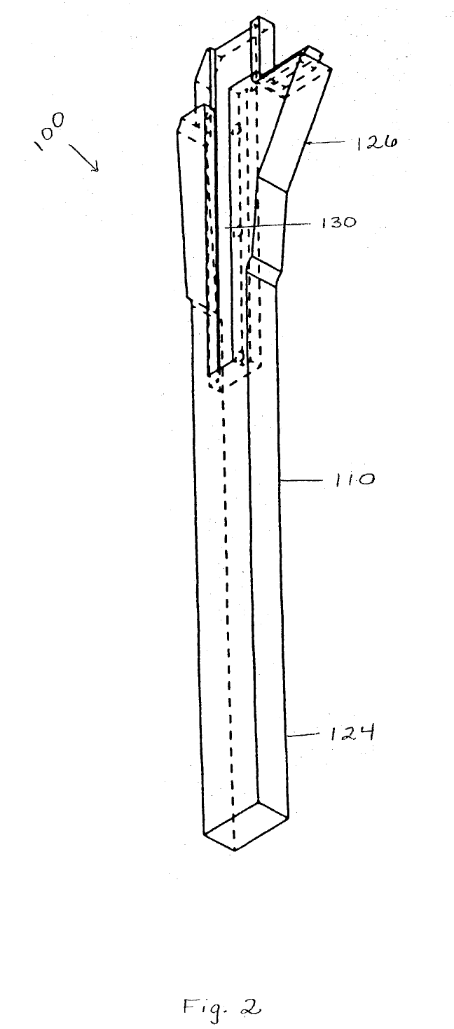

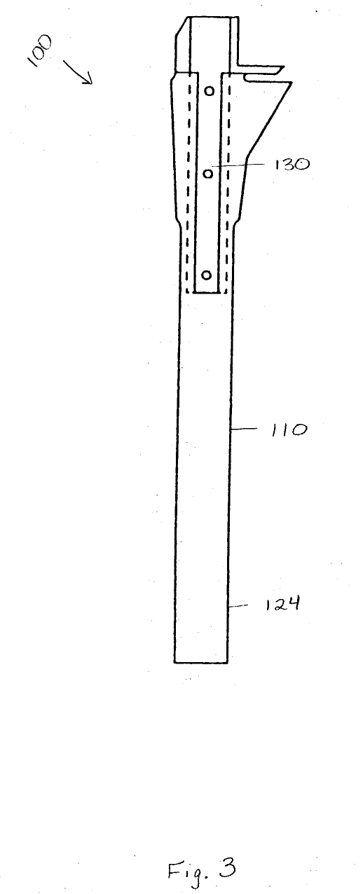

Referring now to FIGS. 2 and 3, the body 110 of the tool 100 is shown in greater detail. The body 110 includes a stem portion 124 which, as shown in FIG. 100, has, for example, a rectangular cross section. The stem portion 124 corresponds to the first portion 112 of the joint 101 (see FIG. 1). The first portion 112 of the joint is the distal stem of the femur. Extending from an end of the stem portion 124 is wider portion 126 of the body 110. The wider portion 126 corresponds to the proximal femur. A slot 130 is positioned longitudinally in the wider portion 126 of the body 110.

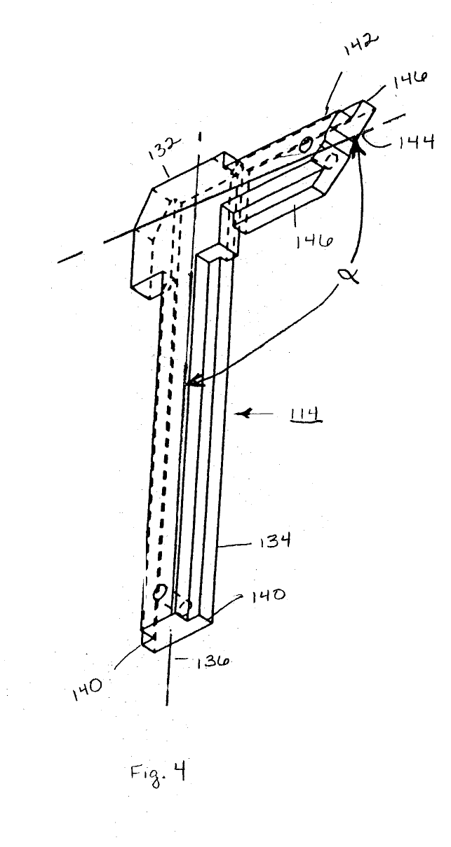

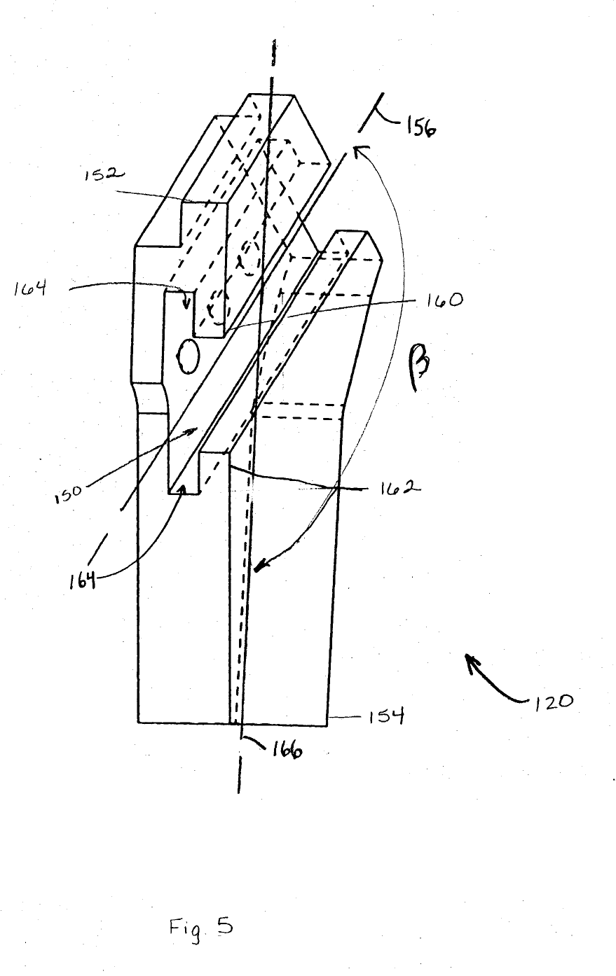

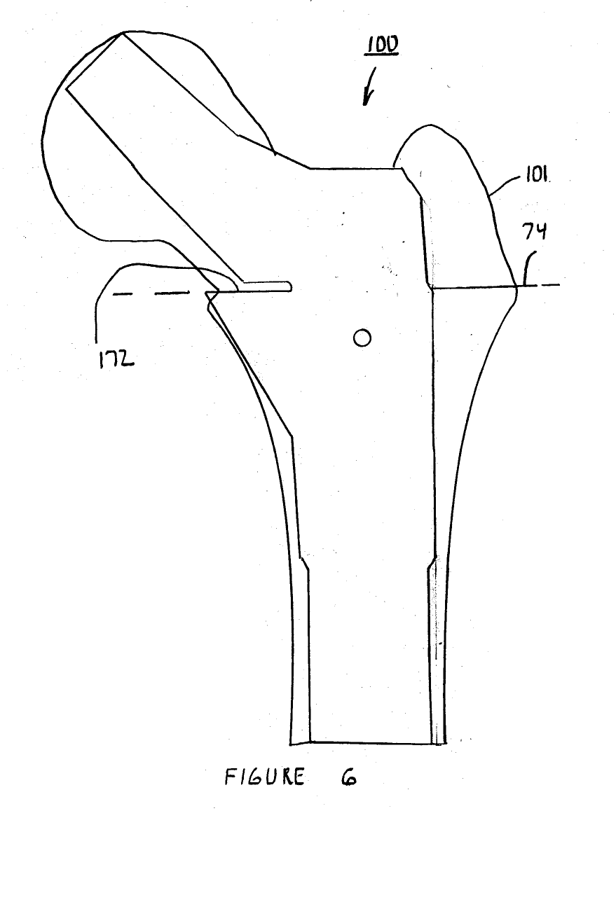

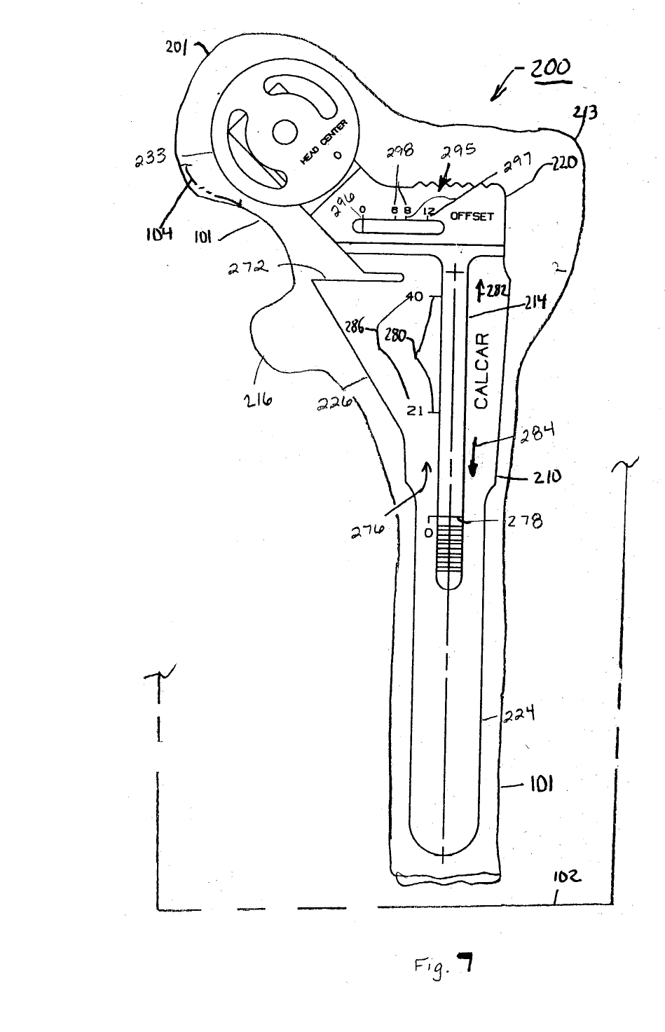

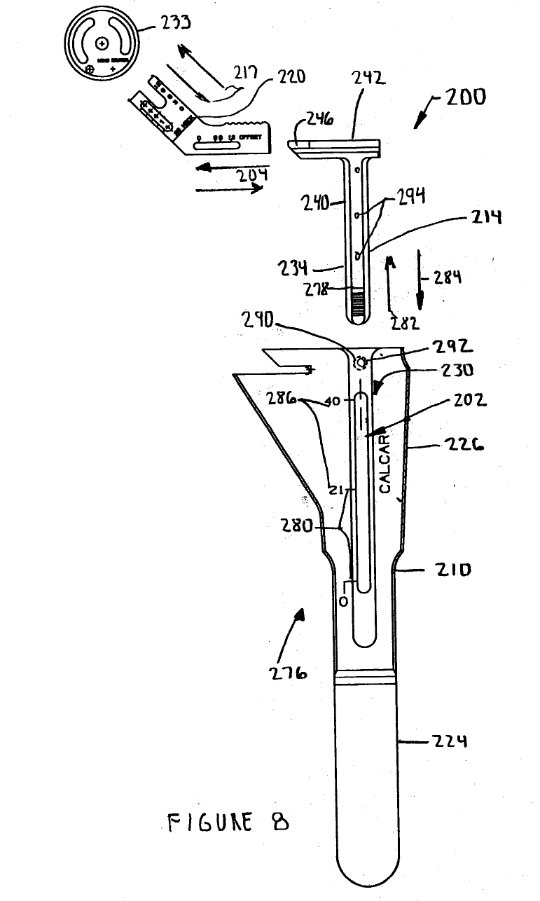

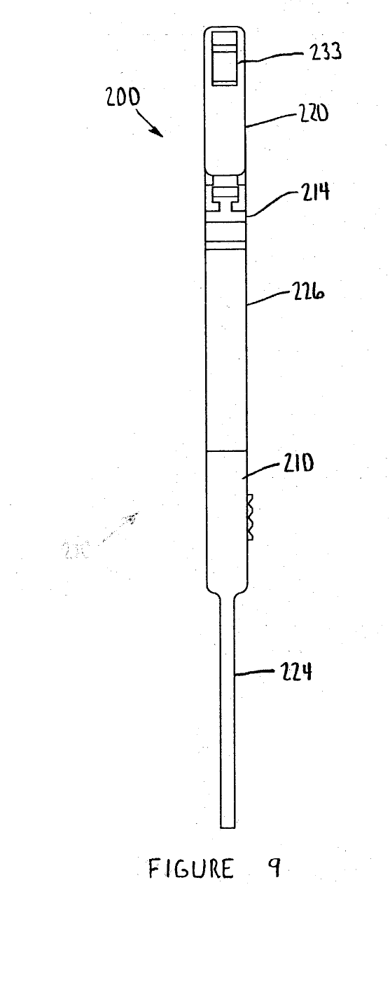

Referring now to FIG. 4, the first segment 114 is shown in greater detail. The first segment 114 includes a first segment base 132.

A calcar slide 134 extends in a first direction from the first segment base 132 along first center line 136. Rails 140 extend along the calcar slide 134 in opposed position from the first center line 136. The rails 140 cooperate with the slot 130 of the body 110 (see FIG. 2).

Referring again to FIG. 4, further extending outwardly from the first segment base 132 is an offset slide 142. The offset slide 142 extends along second center line 144 at, for example, an angle α of, for example, approximately 90 degrees from the first center line 136. Rails 146 similar to the rails 140 extend from the offset slide 142 in a direction normal to the second center line 144. The rails 146 cooperates with slot 150 of the second segment 120 (see FIG. 5).

Referring now to FIG. 5, the second segment 120 is shown in greater detail. The second segment 120 includes a slide portion 152 and a neck portion 154 extending from the slide portion 152. The slide portion 152 includes the slot 150 located along slide centerline 156. While the slot 150 may have any shape capable of guiding and restraining the second segment 120 within the first segment 114, it should be appreciated that the slot 150 may include a proximal lip 160 and a distal lip 162 which form grooves 164 in the slide portion 152. The grooves 164 are sized to matingly fit with the rails 146 of the first segment 114.

The neck 154 extends along neck centerline 166 in a direction defined by β from the slide centerline 156. The angle β preferably corresponds to the neck angle of the prosthesis.

Referring now to FIG. 6, the tool 100 is shown in position over a long bone or femur 101. Thus, as shown in FIG. 6, the tool 100 may be utilized intraoperatively and be positioned over the femur 101, in order to determine the appropriateness of a particular implant prior to initiating bone resection, prior to its removal from its sterile packages and prior to the use of a trial and a trial reduction. Further, the tool 100 may be positioned over the femur 101 and the resection cutting guide surface 172 positioned against the femur 101. A proper resection plane 74 may be determined by an alignment with cutting guide surface 172.

Referring again to FIG. 1, the tool 100 may include indicia 176 to assist in determining the relative position of the body 110 with respect to the first segment 114. The indicia 176 may include a solitary mark 178 on the body 110 and a plurality of first segment marks 180 on the first segment 114. As the first segment 114 is slid along slot 130 of the body 110 in the directions of arrows 182 and 184, the first segment marks 180 may align themselves with the body mark 178. For example, and as shown in FIG. 1, the first segment marks 180 may be identified by reference numbers 186. The reference numbers 186 may refer to a relative or absolute calcar height or may correspond to a particular prosthesis having the appropriate calcar height indicated by the alignment of the body mark 178 with their respective first segment mark 180.

The body and the first segment 114 may be selectably fixedly attachable to each other. For example, the first segment 114 may include a threadable hole or opening 190 to which a screw 192 may be threadably secured. The screw 192 may be selectively tightened and loosened when the first segment 114 is moved into its proper position with respect to the body 110.

To assist in positioning, the first segment 114 with respect to the body 110 in positions corresponding to calcar heights of available prosthesis, the body 110 may include recesses 194 formed centrally with respect to the slot 130 such that the recesses 194 may, when in the proper position along the slot 130 in the direction of arrows 182 and 184, may be in alignment and operatively interacting with the screw 192 to position the first segment mark 180 with the body mark 178 to correspond to available prosthesis with predetermined calcar heights. The screw 192 may include a spring loaded ball end, (not shown) to cooperate with the recessional 194.

Further, the first segment 114 and the second segment 120 may include indicia 195 for determining the relative position of the first segment 114 with respect to the second segment 120. The indicia 195, similarly to the indicia 176, may include a first segment mark 196 positioned on the first segment 114 and a plurality of second segment marks 197 located on the second segment 120. Further, the indicia 195 may include reference numerals 198 associated with the second segment marks 197. The reference numbers 198 may represent the offset positions in absolute or relative terms or may represent particular available prosthetics.

The second segment 120 may be selectively attachable to the first segment 114 by means of, for example, a screw 183, similar to screw 192, which may be threadably secured through hole 181 in the first segment 114. Recesses 185 may be formed in the second segment 120 such that the recesses 185 are positioned so that when aligned with the screw 183, the first segment mark 196 may be aligned with one of the several second segment marks 197.

To assist in the alignment of the tool 100 with respect to either a radiographic image of a femur or an actual femur, the tool 100 may be made of a translucent or transparent material, for example, a plastic.

According to the present invention, and referring to FIG. 7, another embodiment of the present invention is shown as tool 200. The tool 200, similar to tool 100, may be used in conjunction with an image 104 of a joint from a radiograph or x-ray 102. The tool as shown in FIG. 7 is used for arthroplasty for the left hip. Similar to the tool 100 of FIG. 1, the tool 200 may be made of any suitable, durable material and may be, for example, be made of a metal or a plastic. Similar to tool 100, the tool 200 consists of a plurality of parts including, for example, as shown in FIG. 7, a body 210 similar to the body 110 of FIG. 1. As shown in FIG. 7, the body 210 includes a stem portion 224 as well as a wider portion 226. The stem portion 224 is aligned with the distal portion of the femur 101, while the wider portion 226 is aligned with proximal femur.

Similar to the tool 100 of FIG. 1, the tool 200 further includes a first segment 214 which is slidably secured to the body 210. The first segment 214 is slidably movable within the first segment 214 in the direction of arrows 282 and 284. The tool 200 further includes a second segment 220 which is slidably moveable with respect to the first segment 214. Unlike the tool 100, the tool 200 further includes a third segment 233 which is slidably moveable with respect to the second segment 220. The third segment 233 is aligned with head 201 of the femur 101.

Referring now to FIGS. 8, 9, and 11, the body 210 is shown in greater detail. The body 210 includes the distal stem 224 and the wider portion 226. As shown in FIG. 8, the body 210 includes an opening 202 through the body 210. The opening 202 permits viewing of the first segment 214 as it moves up and down in the directions of arrows 282 and 284. Similar to the body 110 of the tool 100 of FIG. 1, the body 210 includes indicia 276 for assisting and determining the relative position of the body 210 with respect to the first segment 214.

For example, as shown in FIG. 11, the indicia 276 include a mark 278 located on the first segment 214 as well as marks in the form of lines 280 on the body 210. Each of the lines 280 may include numbers 286 thereby. The numbers 286 may be indicative of relative distance between the respective lines 280 or may indicate the corresponds to the prosthesis that the tool 200 would indicate is appropriate.

Referring again to FIG. 8, the first segment 214 may be slidably engageable with the body 210 in the direction of arrows of 282 and 284 in any suitable way. For example, the body 210 may include a slot 230, while the first segment 214 may include a calcar slide 234. The slide 230 may slidably fit within the slot 230.

The first segment 214 may be selectively removably secured to the body 210 by, for example, a screw 292 which may be threadably engaged to the body 210 at hole 290 formed in the body 210. The screw 292 may include a spring biased ball shaped contact point (not shown) which may contact recesses 294 formed in the first segment 214. The contact point of the screw 292 may be in contact with the recesses 294 as the first segment 214 is moved in the direction of 282 and 284 making the location of the positions indicated by the numbers 286 more easy to locate, especially when placed by an exposed femur.

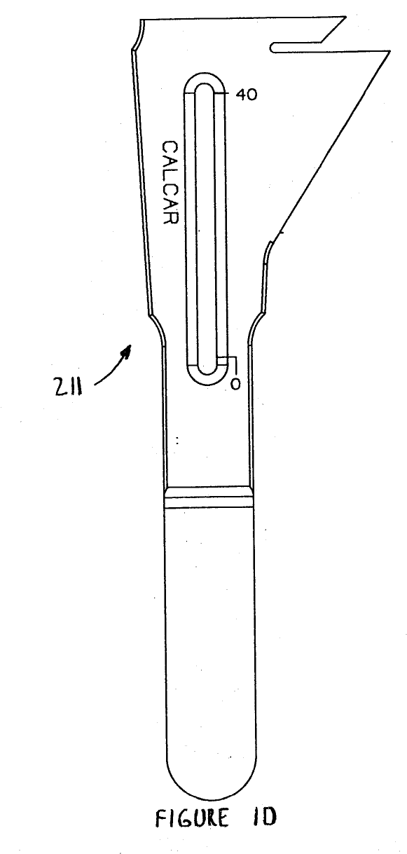

Referring now to FIG. 10, a body 211 is shown which is a mirror image of the body 210 and may be used with a right hip arthroplasty. It should be appreciated that mirror images of first segment 214, second segment 220, and third segment 233 can be used with the body 211 to form a mirror image tool to that of tool 300.

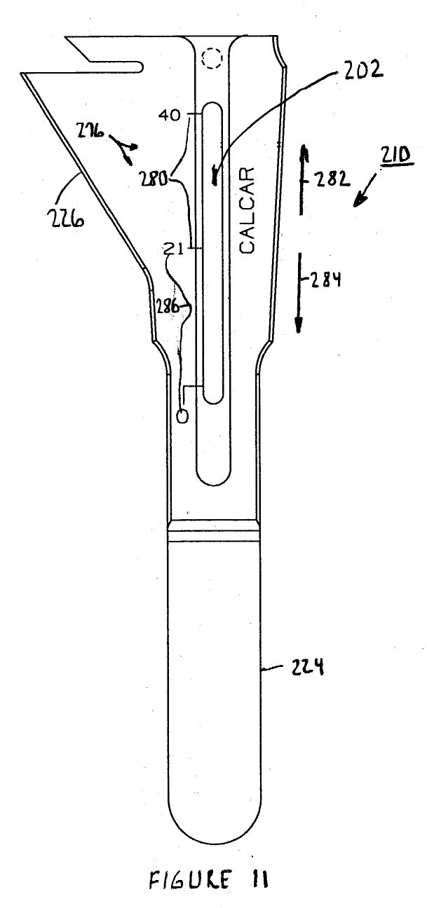

Referring now to FIG. 12, the first segment 214 is shown in greater detail. The first segment 214 is similar to the first segment 114 of the tool 100 of FIG. 1. The first segment 214 includes a central base 232. Extending from the central base 232 along first center line 236 is calcar slide 234. Also extending from the base 232 at an angle αα from the first center line 236 is an offset slide 242 which extends along second center line 244.

Referring again to FIG. 8, the calcar slide 234 slides in the directions of arrows 282 and 284 along slot 230. The calcar slide is guided and retained by rails 240 extending from the calcar slide 234. A mark 278 is located on the calcar slide to assist in determining the relative location of the first segment 214 with respect to the body 210. The offset slide 242 is slidably engaged with the second segment 220 and is guided by rails 246 extending from the offset slide 242.

Referring again to FIG. 8, the second segment 220 is slidably movable in the direction of arrows 204 along offset slide 242.

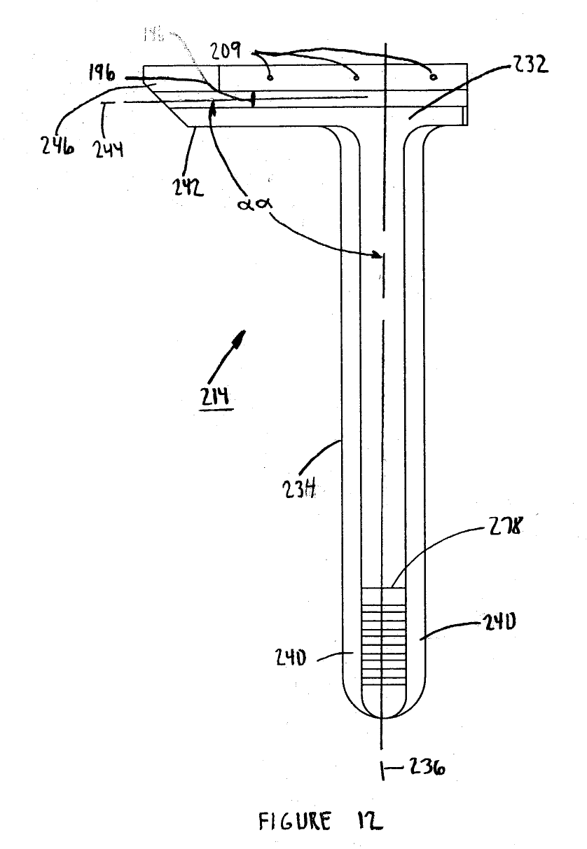

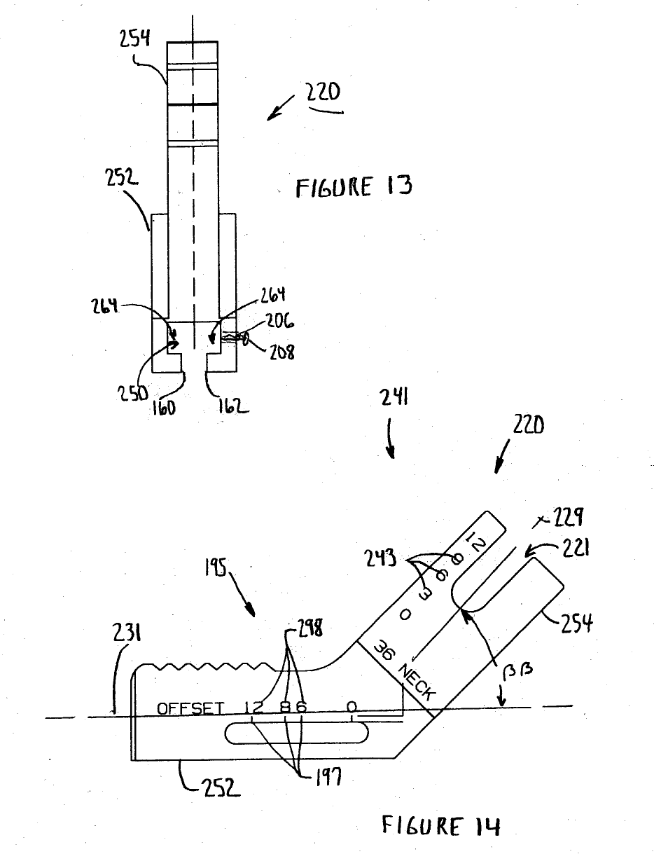

Referring now to FIGS. 13, 14, and 15, the second segment 220 is shown in greater detail. The second segment 220 includes a neck portion 254 and a slide portion 252. The slide portion 252 includes a slot 250 which cooperates with the offset slide 242 of the first segment 214. The slot 250 is defined by lips 260 and 262 which form grooves 264 which mate with rails 246 of the offset slide 242 (see FIG. 8).

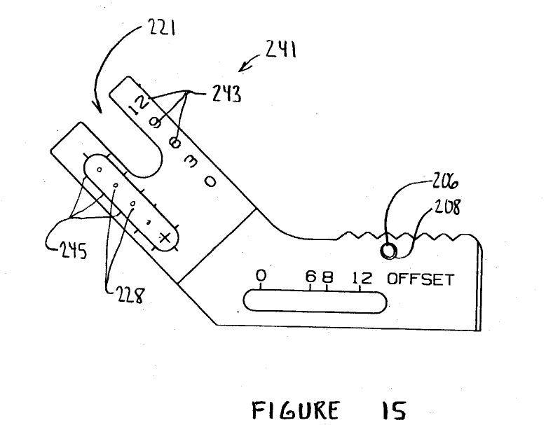

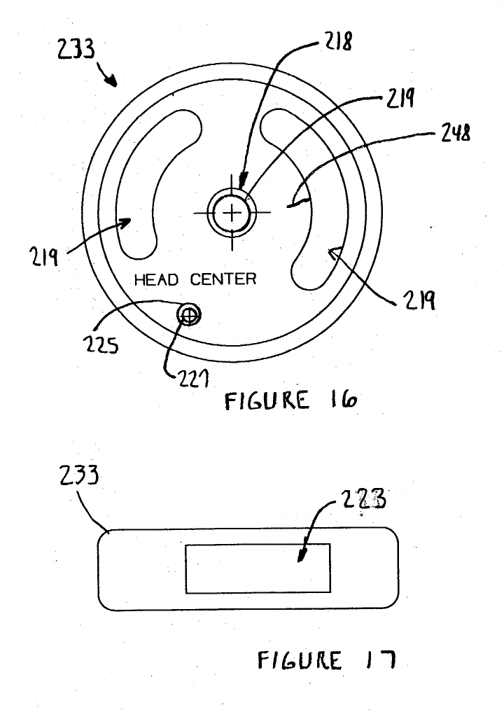

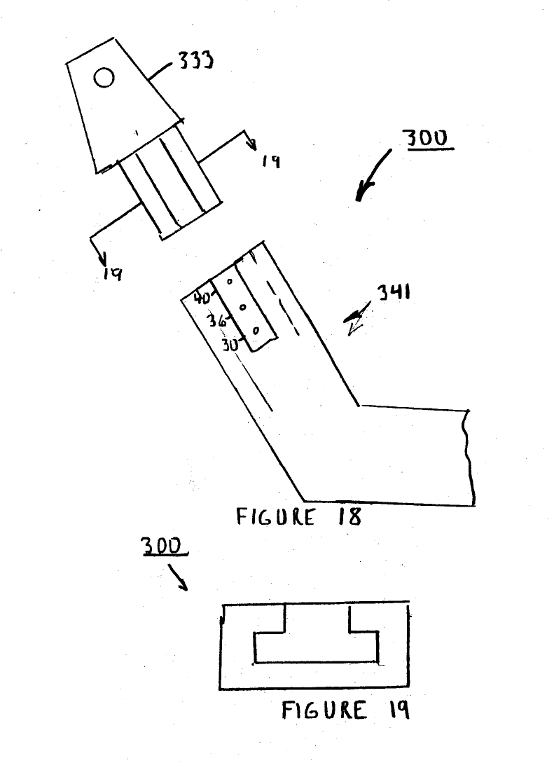

Preferably, and as shown in FIGS. 12 and 14, the tool 200 includes indicia 295 for determining the relative position of the second segment 220 with respect to the first segment 214. The indicia 195 may, for example, be in the form of a plurality of reference marks 197 on one of the first segment 214 and the second segment 220 and a solitary mark on the other of the segments 214 and 220. For example, the second segment 220 may include a plurality of reference marks 197 and the first segment 214 may include a reference mark 278. Reference numerals 298 may be positioned adjacent the reference marks 197 to assist in the relative positioning of the second segment 220 with respect to the first segment 214. The reference numerals 298 may denote relative positions of the reference marks or may correspond to the particular size or number of orthopaedic implants corresponding to the proper implant that the tool 200 would suggest.

Referring to FIGS. 12 through 14, the second segment 220 may be selectively secured to the first segment 214 in any suitable manner. For example, the second segment 220 may include a locking feature for selectively fixably securing the second segment 220 to the first segment 214. For example, the slide portion 252 may include a threaded hole 206 which passes into the slot 250. A screw 208 may be threadably secured to the threaded hole 206. The screw 206 may be similar to the screw 292 used on the body 210. The screw 208 may include a spring-loaded ball tip. The offset slide 242 of the first segment 214 may include a plurality of indents 209 which provide for preset positions in alignment with the reference numerals 298.

Referring again to FIG. 8, the third segment 233 is slidably connected to second segment 220 and is permitted to move in the direction of arrows 217.

Referring now to FIGS. 14, 15, 16 and 17, the third segment 233 is shown in greater detail. The third segment 233 has a generally cylindrical shape and as shown in FIG. 16 includes a central hole 218 as well as two arcuate openings 219. The third segment 233 may be slidably secured to the neck portion 254 of the second segment 220 in a suitable fashion. For example, a pin 219 may be fittably secured to the hole to 218 in the third segment 233. The pin 219 may be slidably fitted to opening 221 in the neck portion 254 of the second segment 220.

Alternatively, or in addition to the use of the pin 219, the third segment 233 may include a central slot 223 into which the neck portion 254 may be slidably fitted.

The third segment 233 may be selectably fixably secured to the second segment 220 in any suitable fashion. For example, the third segment 233 may include a threaded hole 225 to which screw 227 may be threadably attached. The screw 227 may be similar to the screw 208 and may include a spring biased spherical tip. To provide for preset positions, the second member 220 may include a plurality of indents 228 which may be aligned with the screw 227 to provide for preselected positions. The preselected positions may be in alignment with reference numerals 243.

Referring to FIGS. 14 through 16, the neck portion 254 of the second segment 220 may extend from the slide portion 252 of the second segment 220 along a neck center line 229 which extends at a angle ββ from the slide center line 231 of the slide portion 252.

To assist in observing the position of the third segment 233 with respect to the second segment 220, the second segment 220 and/or the third segment 233 may include indicia 241 for determining the relative location of the third segment 233 with respect to the second segment 220. For example, the indicia 241, may be in the form of a plurality of marks 245 on the neck portion 254 of the second segment 220 and a solitary mark 248 located on the third segment 233. In addition, the indicia 241 may include reference numbers 243 which may be positioned in alignment with the marks 245 and which may correspond to particular spacing distance between the adjacent marks 245 or correspond to the suggested implant that corresponds with the particular settings determined on the tool 200.

Referring now to FIGS. 18 and 19, an alternate embodiment of the present invention is shown as tool 300. Tool 300 is similar to the tool 200 of FIGS. 7 through 17 except that in place of the third segment 233 of the tool 200, the tool 300 includes a third segment 333 which has a generally tapered shape and to correspond with the tapered shape of a neck of a prosthetic stem. It should be appreciated that tool 300 may further include indicia 341 similar to the indicia 195 of the second segment 220 of the tool 200.

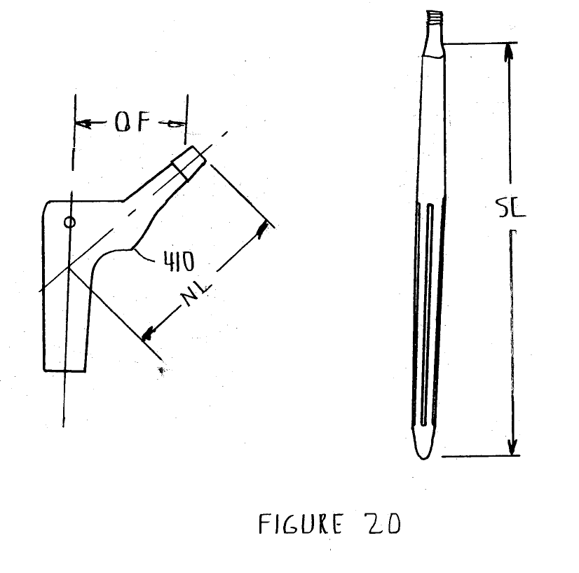

Referring now to FIG. 20, a two-piece prosthesis 400 is shown for use in conjunction with the tool 100. The prosthesis 400 includes a proximal body 410 and a distal stem 412. It should be appreciated that the proximal body 410 may have a variety of shapes including a variety of offset lengths of and neck lengths NL. Similarly, the distal stem 412 may have a variety dimensions including a variety of stem lengths SL.

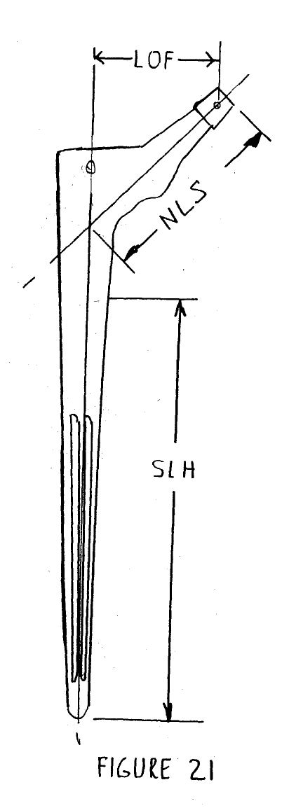

Referring now to FIG. 21, a monolithic prosthesis 450 is shown. It should be appreciated that the prosthesis may have a variety of shapes including a variety of lateral offsets LOF, neck lengths NLS and a variety of stem lengths SLH.

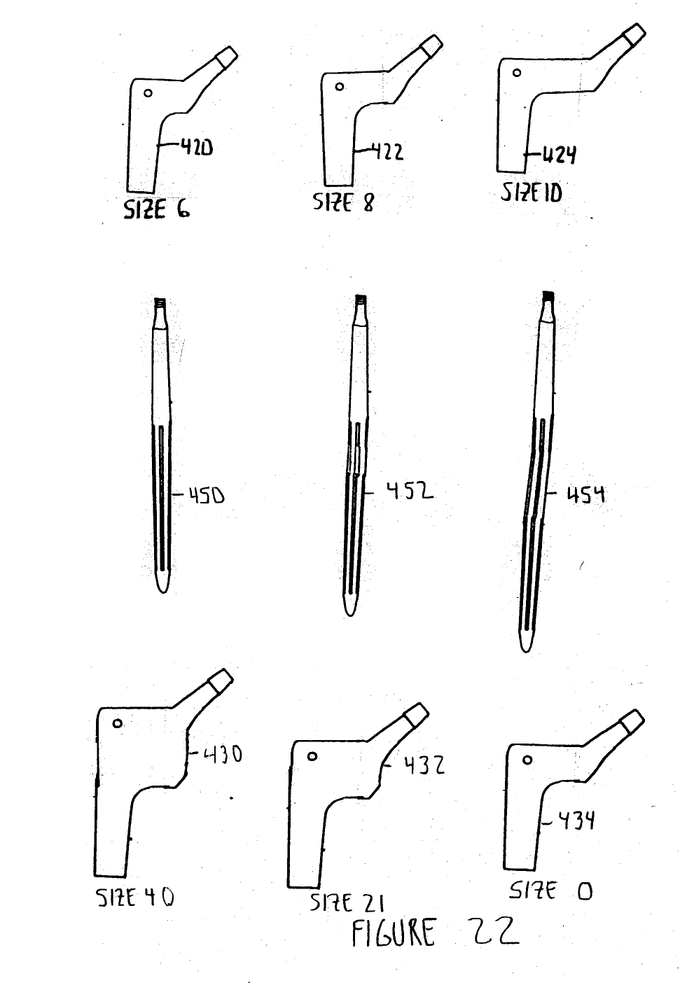

Referring now to FIG. 22, a variety of proximal bodies and distal stems for use with the prosthesis 400 is shown. The proximal bodies include a size 6 proximal body 420, a size 8 proximal body 422, and a size 10 proximal body 424. The proximal bodies also include a size 40 proximal body 430, size 21 proximal body 432, and a size 0 proximal body 434. It should be appreciated that the preferred distal stem of the short stem 450, the medium length stem 452 and the long stem 454 could be selected with the use of the template 100 of the present invention (see FIG. 1) by using for example a sliding arm 199 (shown in phantom) extendable from the base 110 including indicia (not shown) corresponding to one of the three possible stems.

Referring now to FIGS. 1 and 22, the reference numerals 186 on the tool 100 correspond to the proximal bodies 434, 432, and 434, respectively. Thus, as shown in FIG. 1, when the appropriate calcar height is a size 0, i.e., when the reference numeral 186 is align with body mark 178, the appropriate proximal body is proximal body 434.

Similarly, referring to FIG. 1 and FIG. 22, when the appropriate offset is a size 8, i.e., when the reference numeral 198 on the second segment 120 is aligned with the first segment marked 196, the reference numeral 8 corresponds to the choice of selecting the proximal body 422.

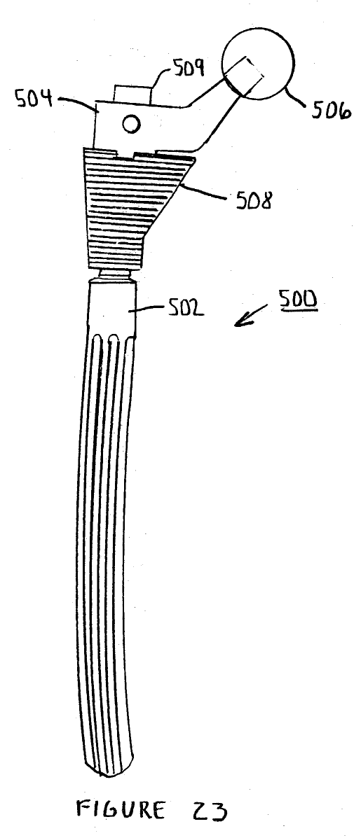

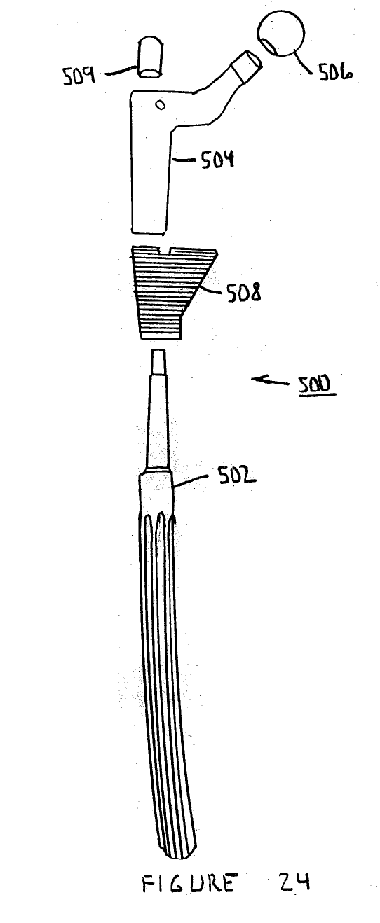

Referring now to FIGS. 23 and 24, a multiple piece prosthetic hip femoral component 500 is shown. The femoral component 500 includes a distal stem 502 which is connected to a proximal body 504. A head 506 is connected to the proximal body 504. A sleeve 508 is fitted over the proximal body 504. A nut 509 is used to secure the proximal body to the distal stem 502.

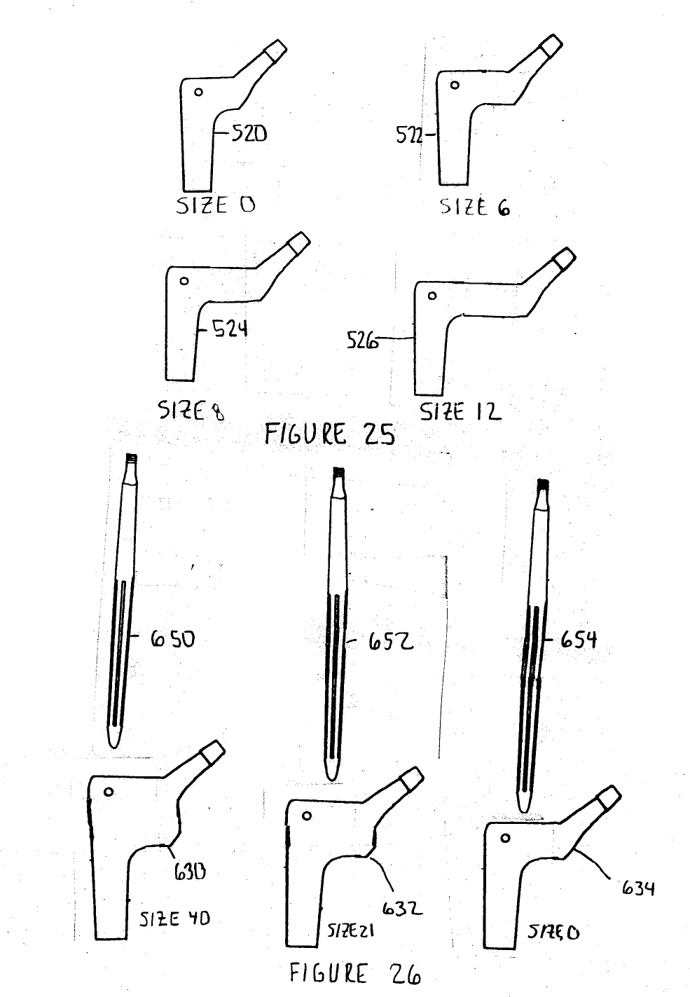

Referring now to FIGS. 25, 26, and 27, a plurality of distal stems and proximal bodies, and heads are shown for use in conjunction with the tool 200 of FIGS. 7 through 17. For example, in referring to FIGS. 7 and 25, the proximal body may be selected with the proper offset by doing the proper offset from the second segment 220 of the tool 200 and observing the proper number 298. By referring to the proper number 298, the corresponding appropriate proximal body may be selected. For example, for offset 0, the proximal body 520 would be selected, for the offset size 6, the proximal body 522 would be selected, for the offset size 8, the proximal body 524 would be selected and for the offset size 12, the proximal body 526 would be selected. It should be appreciated that the preferred distal stem of the short stem 650, the medium length stem 652 and the long stem 654 could be selected with the use of the template 100 of the present invention (see FIG. 1) by using for example a sliding arm 199 (shown in phantom) extendable from the base 110 including indicia (not shown) corresponding to one of the three possible stems.

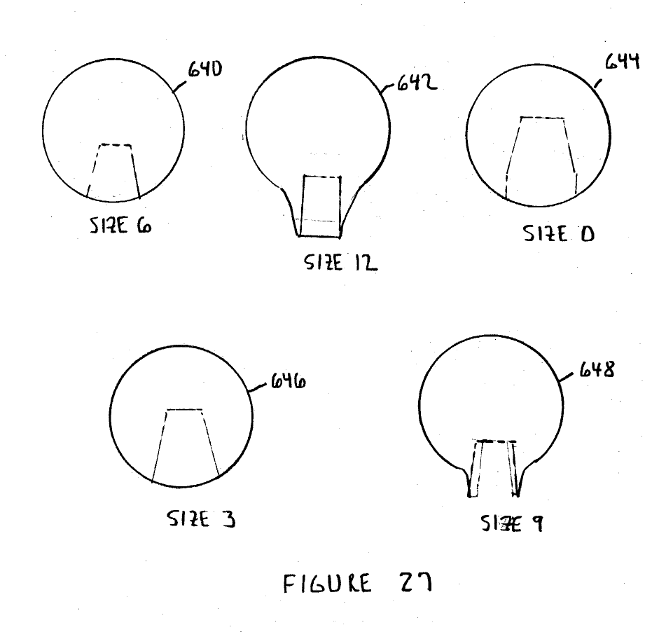

Referring now to FIGS. 7 and 26, the appropriate proximal body stem would be selected by observing the calcar size recommendation from the tool 200 observed from the reference numeral 286 on the body. For example, for reference numeral 40, the proximal body 630 would be selected. For the calcar size 21, the proximal body 632 would be selected and for the calcar size 0, the proximal body 634 would be selected.

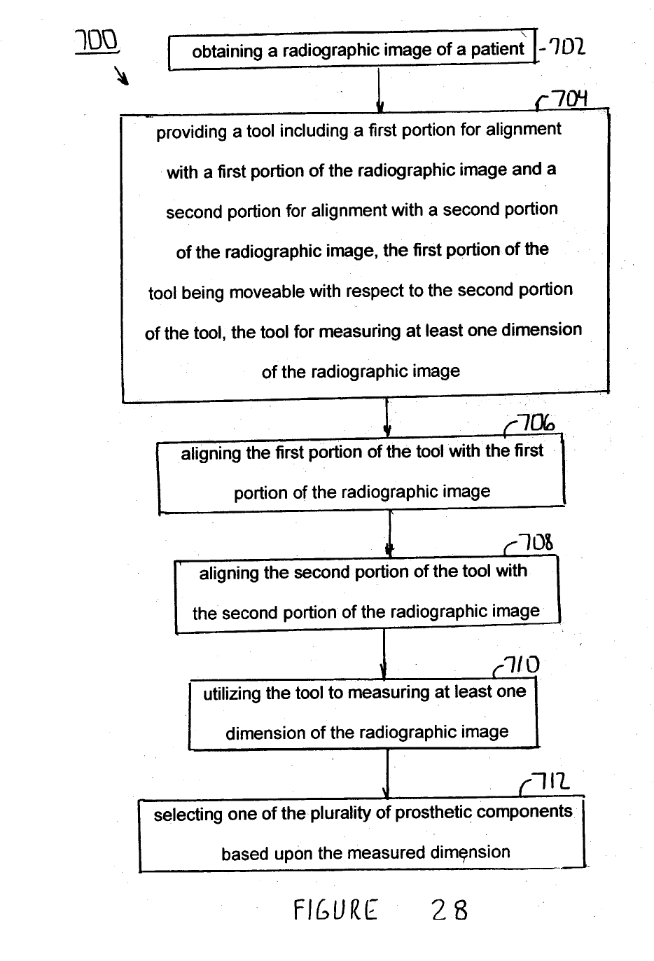

Referring now to FIG. 27 and FIG. 14, the appropriate head would be selected based on the reference numbers 243 selected from the indicia 241 as shown in FIG. 14. For example, for a head center number 6, the head 640 would be selected, for the head center 12, the head 642 would be selected, for the head center 0, the head 644 would be selected, for the head center 3, the head 646 would be selected, and for the head center number 9, the head 648 would be selected.

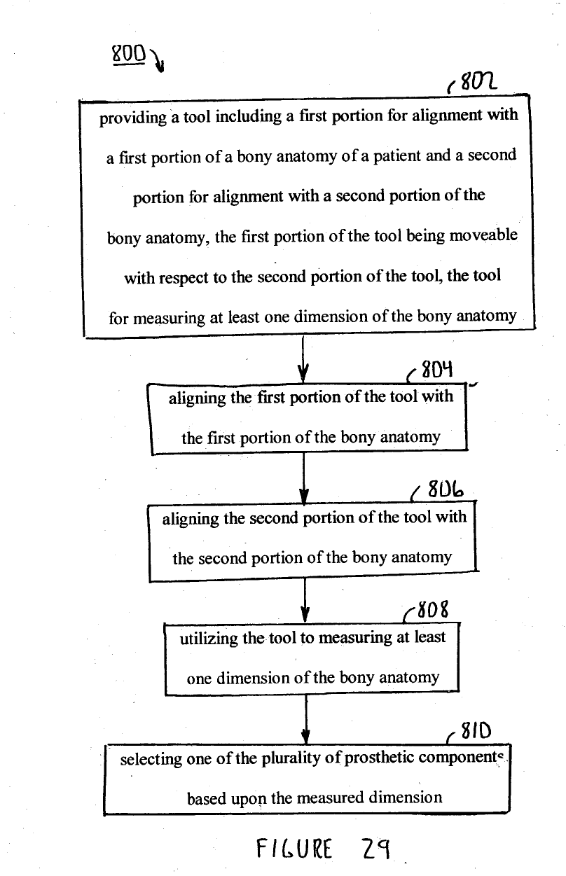

Referring now to FIG. 28, a method 700 for selecting one of a plurality of prosthetic components for use in joint arthroplasty is shown. The method 700 includes the first step 702 of obtaining a radiographic image of a bony anatomy of a patient. The method 700 includes the second step 704 of providing a tool including a first portion for alignment with a first portion of the radiographic image and a second portion for alignment with a second portion of the radiographic image, the first portion of the tool being moveable with respect to the second portion of the tool, the tool for measuring at least one dimension of the radiographic image. The method 700 includes the third step 706 of aligning the first portion of the tool with the first portion of the radiographic image. The method 700 includes the fifth step 708 of aligning the second portion of the tool with the second portion of the radiographic image. The method 700 includes the sixth step 710 of utilizing the tool to measure at least one dimension of the radiographic image and the seventh stem 712 of selecting one of the plurality of prosthetic components, based upon the measured dimension.

Referring now to FIG. 29, a method 800 for selecting one of a plurality of prosthetic components for use in joint arthroplasty is shown. The method 800 includes the first step 802 of providing a tool including a first portion for alignment with a first portion of a bony anatomy of a patient and a second portion for alignment with a second portion of the bony anatomy, the first portion of the tool being moveable with respect to the second portion of the tool, the tool for measuring at least one dimension of the bony anatomy. The method 800 includes the second step 804 of aligning the first portion of the tool with the first portion of the bony anatomy. The method 800 includes the third step 806 of aligning the second portion of the tool with the second portion of the bony anatomy. The method 800 includes the fourth step 808 of utilizing the tool to measure at least one dimension of the bony anatomy and the sixth stem 810 of selecting one of the plurality of prosthetic components, based upon the measured dimension.

Although the present invention and its advantages have been described in detail, it should be understood that various changes, substitutions, and alterations can be made therein without departing from the spirit and scope of the present invention as defined by the appended claims.

We claim:

1. A tool for aiding in the selection of an appropriately sized implant for use in performing joint arthroplasty, the tool for cooperation with at least one of an anatomical joint an image of an anatomical joint, said tool comprising:
a body for alignment with a first portion of the anatomical joint; and
a segment, said segment being moveable with respect to said body, said segment for alignment with a second portion of the anatomical joint, said body and segment adapted for measuring the relative positions of the first and second portions of the anatomical joint for use in the selection an appropriately sized implant.

2. The tool of claim 1, further comprising a second segment, said segment being moveable with respect to one of said body and said first mentioned segment, said third segment for alignment with a third portion of the anatomical joint.

3. The tool of claim 2, further comprising a third segment, said segment being moveable with respect to one of said body, said first mentioned segment and said second mentioned segment, said third segment for alignment with a fourth portion of the anatomical joint.

4. The tool of claim 2, wherein said body defines a channel therein, said first mentioned segment being guidable within the channel.

5. The tool of claim 1, wherein at least one of said body and said first mentioned segment include indicia thereon of determining the relative position of said body with respect to said first mentioned segment.

6. The tool of claim 1, wherein at least one of said body and said first mentioned segment includes a portion thereof which is translucent.

7. The tool of claim 1, wherein at least one of said body and said first mentioned segment are selectably fixedly attachable to each other.

8. The tool of claim 1, wherein the image is a radiographic image of an anatomical joint.

9. The tool of claim 1:
wherein said body includes a body indicia thereon and;
wherein said first mentioned segment includes a plurality of segment indicia thereon at least a portion of said segment indicia corresponding to a recommended size of prosthetic implant.

10. The tool of claim 10, wherein the recommended dimension of prosthetic implant is one of the calcar height, the offset and the neck length.

11. A tool for use in measuring at least one of certain dimensions for aiding in the selection an appropriate one of a plurality of femoral components of a hip implant for use in performing hip arthroplasty, the tool for cooperation with at least one of an anatomical joint and a radiographic image of an anatomical joint, said tool comprising:
a body for alignment with a first portion of the anatomical joint; and
a segment, said segment being moveable with respect to said body, said segment for alignment with a second portion of the anatomical joint, whereby when said body is in alignment with the first portion and when said segment is in alignment with the second portion the tool is used to measure one of the certain dimensions.

12. The tool of claim 11, wherein the one of the certain dimensions is the calcar height.

13. The tool of claim 11, further comprising:
a second segment, said segment being moveable with respect to said first mentioned segment, said first mentioned segment for use in measuring the calcar height and said second segment for use in measuring the offset.

14. The tool of claim 13, further comprising a third segment, said third segment being moveable with respect to said second segment, said third segment for use in measuring the neck length.

15. The tool of claim 11, wherein at least one of said body and said first mentioned segment include indicia thereon for measuring at least one of the certain dimensions.

16. The tool of claim 11, wherein at least one of said body and said first mentioned segment includes a portion thereof which is translucent.

17. The tool of claim 11, wherein at least one of said body and said first mentioned segment are selectably fixedly attachable to each other.

18. The tool of claim 11:
wherein said body includes a body indicia thereon and;
wherein said first mentioned segment includes a plurality of segment indicia thereon at least a portion of said segment indicia corresponding to a recommended size of prosthetic implant.

19. A method for selecting one of a plurality of prosthetic components for use in joint arthroplasty comprising:
obtaining a radiographic image of a bony anatomy of a patient;
providing a tool including a first portion for alignment with a first portion of the radiographic image and a second portion for alignment with a second portion of the radiographic image, the first portion of the tool being moveable with respect to the second portion of the tool, the tool for measuring at least one dimension of the radiographic image;
aligning the first portion of the tool with the first portion of the radiographic image;
aligning the second portion of the tool with the second portion of the radiographic image;
utilizing the tool to measure at least one dimension of the radiographic image; and
selecting one of the plurality of prosthetic components, based upon the measured dimension.

20. A method for selecting one of a plurality of prosthetic components for use in joint arthroplasty comprising:
providing a tool including a first portion for alignment with a first portion of a bony anatomy of a patient and a second portion for alignment with a second portion of the bony anatomy, the first portion of the tool being moveable with respect to the second portion of the tool, the tool for measuring at least one dimension of the bony anatomy;
aligning the first portion of the tool with the first portion of the bony anatomy;
aligning the second portion of the tool with the second portion of the bony anatomy;
utilizing the tool to measuring at least one dimension of the bony anatomy; and
selecting one of the plurality of prosthetic components, based upon the measured dimension.
